(19) United States
(12) Patent Application Publication
(10) Pub. No.: US 2013/0158349 A1
(43) Pub. Date: Jun. 20, 2013

(54) INSERTION AND EXTRACTION ASSISTING DEVICE AND ENDOSCOPE SYSTEM

(71) Applicants: Tsuyoshi ASHIDA, Kanagawa (JP); Takayuki NAKAMURA, Kanagawa (JP); Shinichi YAMAKAWA, Kanagawa (JP); Yasunori OHTA, Kanagawa (JP); Rick Cornelius, Wayzata, MN (US); Maureen P. Sheridan, Eagan, MN (US)

(72) Inventors: Tsuyoshi ASHIDA, Kanagawa (JP); Takayuki NAKAMURA, Kanagawa (JP); Shinichi YAMAKAWA, Kanagawa (JP); Yasunori OHTA, Kanagawa (JP); Rick Cornelius, Wayzata, MN (US); Timothy P. SHERIDAN, Eagan, MN (US)

(73) Assignee: FUJIFILM CORPORATION, Tokyo (JP)

(21) Appl. No.: 13/543,316

(22) Filed: Jul. 6, 2012

Related U.S. Application Data
(60) Provisional application No. 61/505,692, filed on Jul. 8, 2011.

(51) Int. Cl. A61B 1/00; A61B 1/04

(52) U.S. Cl. CPC A61B 1/00133; A61B 1/04
USPC 600/109; 600/114

(57) ABSTRACT

An insertion and extraction assisting device of an endoscope has a promotion unit attached on the front end side of the endoscope and a transmission section whose one end is connected to a promotion unit, and the other end is drawn along the endoscope, and an outside unit. The other end of the transmission section is connected to the outside unit, and rotational force or a turn signal from the outside unit is transmitted to the promotion unit, so that driving force is generated by the promotion unit. When the other end of the transmission section is connected to, the outside unit reads an ID code expressing a kind of the promotion unit and controls the rotational force or the turn signal to send to the promotion unit.

### BACKGROUND OF THE INVENTION

1. Field of the Invention

This invention relates to an insertion and extraction assisting device for assisting the insertion of an endoscope into a body cavity, and an endoscope system.

2. Description of the Related Art

Endoscopes are widely used for observation in body cavities. This kind of endoscope includes an insertion section to be inserted into a body cavity, and an operation unit for manipulating this insertion section. Extensive experience is required for insertion of the endoscope into the body cavity. Especially, the procedure of inserting the endoscope in a large intestine is difficult since the large intestine includes parts which are not fixed to the body cavity, such as sigmoid colon and transverse colon. As listed in Japanese Patent Laid-Open Application Publication No. 2008-272302 and U.S. Pat. No. 6,971,990, it is known that there is an insertion and extraction assisting device which facilitates insertion operation of the endoscope in the body cavity by operating a driving member, for example a turn cylinder or a circulation movable body, at the front end side of an insertion section of the endoscope.

As the insertion and extraction assisting device, there are a built-in type incorporated in the inside of the insertion section and a wearing type that a promotion unit having self-promotion function is attached to the insertion section. It is necessary for the built-in type to change overall structure of the insertion section, and production cost of the overall endoscope rises. In addition, because not only the maintenance of the endoscope becomes troublesome, but also insertion assistance function is uniformly fixed to every endoscope, it is inconvenient in considering that there are various insertion operations depending on cases.

In contrast, because the wearing type can just apply to the conventional endoscope, there are many advantages such as that an operator can select and use a promotion unit depending on case if several kinds of the units varying in size and function are prepared for the same endoscope. In case a small motor can be incorporated in the promotion unit, a turn signal of the motor is sent to the promotion unit from an outside unit. In case the promotion unit cannot have a built-in motor, mechanical rotational force of the motor incorporated in an outside unit is sent to the promotion unit through torque wires or the like. Accordingly, as to the wearing type, there are a case that the promotion unit with the built-in motor is operated with the outside unit without the motor, and a case that the promotion unit without the motor is operated with the outside unit with the built-in motor. In either case, by commonizing a transmission section which electrically or mechanically connects between the promotion unit and the outside unit to transmit rotational force or a turn signal, there becomes a merit that the outside unit can be commonly used for different types of promotion units even though it is limited inside a rotational force transmission system or a turn signal transmission system.

However, when the kinds of the promotion units increase as described above, it becomes hard to be recognized that what unit is appropriate to be used under what condition. And in case it was connected with the outside unit, there is a possibility to have to readjust number of revolutions and a maximum torque of the motor to appropriate range depending on the kind of the promotion unit and the case, therefore it takes effort with complicatedness to confirm an appropriate set range on specifications each time. In addition, when the kinds of applicable endoscopes further increase, it cannot be always said that all promotion units can be used in all endoscopes appropriately. For example, in case a specific promotion unit is attached to a distal end portion of a specific endoscope, when the outer diameter of the endoscope is too narrow, the promotion unit cannot be put on closely and it is possible to occur a falling off accident.

### SUMMARY OF THE INVENTION

The purpose of the present invention is to provide an insertion and extraction assisting device which distinguishes a kind of a promotion unit mounted in an endoscope automatically, and sends rotational force or a turn signal to the promotion unit from an outside unit to meet the drive condition that adapted to the kind of the promotion unit.

To achieve the above objects, an insertion and extraction assisting device of the present invention includes a promotion unit attached on the front end side of an insertion section, an outside unit which generates rotational force or a turn signal to drive the promotion unit, and a transmission section to transmit the rotational force or the turn signal from the outside unit to the promotion unit. The promotion unit has a driving member which turns by the rotational force from outside or a driving member which turns via a motor based on the turn signal from outside, and when the driving member turns, driving force is generated at the front end side of the insertion section. The transmission section is connected to the promotion unit at one end side and provided with an input part which is at the other end side and is connected to a coupler of the outside unit, so that the promotion unit is connected to the outside unit. A cord given section is provided in the other end side of the transmission section, and a cord representing the kind of the promotion unit is given to the cord given section. A reader is incorporated in the outside unit and reads the cord of the promotion unit from the cord given section while being connected to the coupler. A control section in the outside unit controls the rotational force or the turn signal sent to the promotion unit from the outside unit according to the read code.

The endoscope system of the present invention includes an insertion and extraction assisting device having a promotion unit, a transmission section and an outside unit, an endoscope, and a processor unit. The processor unit displays image on a monitor based on an image signal from the endoscope, and outputs an identification code corresponding to a kind of the endoscope connected to the processor unit. A reader is provided to the outside unit of the insertion and extraction assisting device and reads a cord representing a kind of the connected promotion unit. A control section of the outside unit distinguishes whether a combination of a code read in the above readers and an identification code input from the processor unit is appropriate.

According to the present invention, when an optimum one is selected from a great variety of the promotion units prepared beforehand, depending on a purpose or a case, to be attached to the insertion section of the endoscope, since rotational force or a turn signal from the outside unit is controlled automatically so that the promotion unit operates within the limit of the drive condition adapted to its kind, even if an operator is inexperienced in handling of the insertion and extraction assisting device, the operator does not operate the promotion unit abnormally and can insert the endoscope in the body cavity safely.

In addition, according to the endoscope system of the present invention, when the endoscope is combined with the insertion and extraction assisting device to perform insertion operation, since it is automatically distinguished whether the combination of the kind of the endoscope and the promotion unit is appropriate, it can be surely prevented that a wrong combination is used for the operation and an unexpected accident occurs.

### BRIEF DESCRIPTION OF THE DRAWINGS

The above purposes and advantages can be easily understood by a skilled person by reading the detailed description of preferable embodiments of the present invention with reference to attached drawings:

FIG. 1 is a schematic diagram indicating a structure of an endoscope system of the present invention;

FIG. 2 is a perspective view indicating an appearance of a promotion unit of an insertion and extraction assisting device;

FIG. 3 is a longitudinal sectional view of the promotion unit;

FIG. 4 is an explanatory drawing indicating a connecting portion with the promotion unit and a coupler of a drive unit;

FIG. 5 is a block diagram indicating an electrical constitution of the drive unit; and

FIG. 6 is a flow chart indicating the steps of drive control of the promotion unit.

### DESCRIPTION OF THE PREFERRED EMBODIMENTS

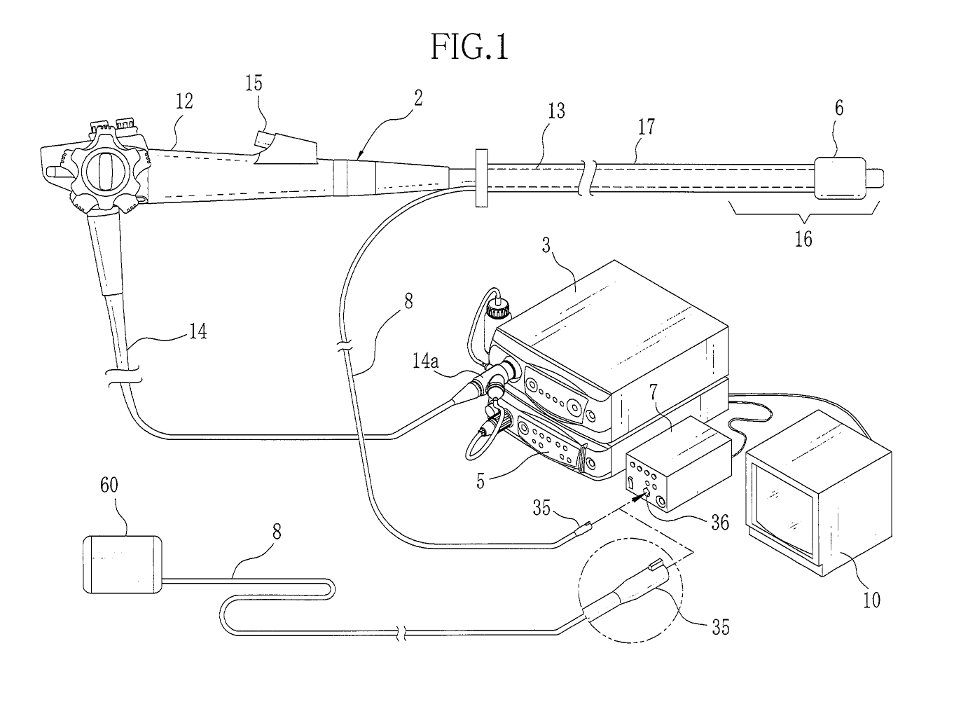

As illustrated in FIG. 1, an endoscope system of the present invention includes an endoscope 2, a light source unit 3 and a processor unit 5 connected to the endoscope 2, a promotion unit 6 attached to the front end side of an insertion section of the endoscope 2, an outside unit 7 which drives the promotion unit 6, and a monitor 10 electrically connected to the processor unit 5. The promotion unit 6 and the outside unit 7 are connected by a torque wire 8 which transmits rotational force.

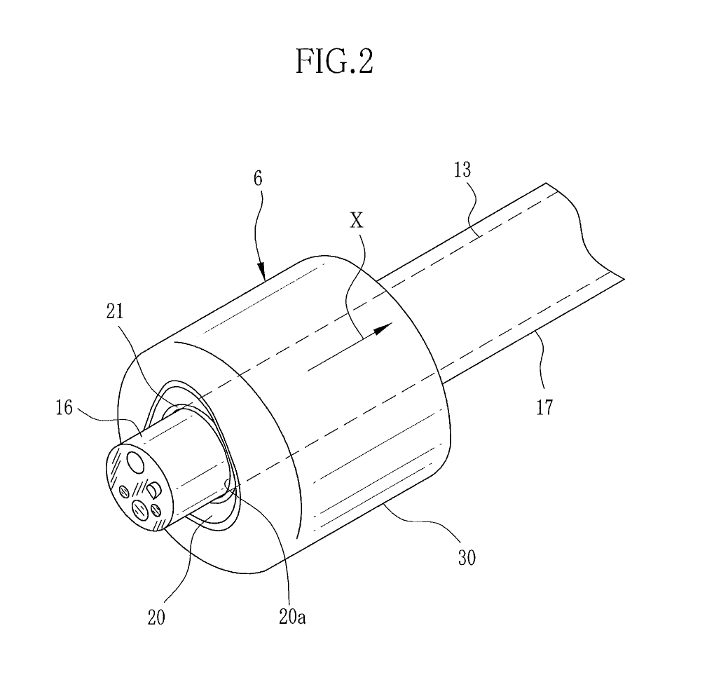

The endoscope 2 has an operation unit 12, an insertion section 13 and a universal cord 14. In the operation unit 12, operation buttons and operation dials to perform angle operation and change operation of air supply and exhaust/water supply and drainage are provided, and a forceps channel opening 15 to which forcipes are inserted is provided. A front end side 16 of insertion section 13 can be widely curved in response to a curve operation with the operation unit 12. The endoscope 2 is the direct viewing type, and as illustrated in FIG. 2, well-known imaging window, illumination window, air supply/water supply opening, and so on are provided on the end face of the front end side 16. In addition, the proximate end side of insertion section 13 is a flexible tube section, and the operation unit 12 is connected to this.

A light guide fiber and an electrical signal line are incorporated in the universal cord 14, and are inserted through the operation unit 12 into the inside of the insertion section 13. The light guide fiber is connected to the light source unit 3 through a connector 14a, leads illumination light from a lamp incorporated in the light source unit 3 to the front end side 16 of the insertion section and radiates the light from an illumination window. For example a CMOS-type image sensor is incorporated inside the front end side 16, so that an optical image obtained through an objective optical system inside the imaging window is imaged. The obtained video signal is input into the processor unit 5 through a signal line inside the insertion section 13, the operation unit 12 and the universal cord 14. The image in the body cavity is displayed to the monitor 10 based on this video signal. The operator performs the insertion operation while observing an image on the monitor 10 or observes an observation site. In addition, from the universal cord 14, an identification code representing a kind of the connected endoscope 2 is input into the processor unit 5. By using the identification code input in this way, the processor unit 5 becomes able to send for example a control signal to the light source unit 3 and perform light controls of the illumination light so as to be suitable for the endoscope 2 in service.

In case of an endoscope for observing a large intestine, an insertion and extraction assisting device is used particularly for the purpose of easy insertion operation into a sigmoid colon or a transverse colon. The insertion and extraction assisting device is composed of the promotion unit 6 attached to the front end side 16 of the insertion section of the endoscope 2, the torque wire 8 which transmits rotational force to produce driving force of the promotion unit 6, and the outside unit 7 which gives the rotational force to the torque wire 8. A motor is provided inside of the outside unit 7, the turn of the motor is transmitted to the promotion unit 6 through the torque wire 8, and a driving mechanism incorporated in the promotion unit 6 is activated to generate the driving force in a forward or backward direction in the front end side 16 of the insertion section 13. The torque wire 8 has the structure in which a flexible core wire 8a whose torsional deformation can be ignored is inserted in a cover tube having flexibility. A flexible overtube 17 is connected to the rear end side of the promotion unit 6, and the torque wire 8 is drawn outside through inside of the overtube 17.

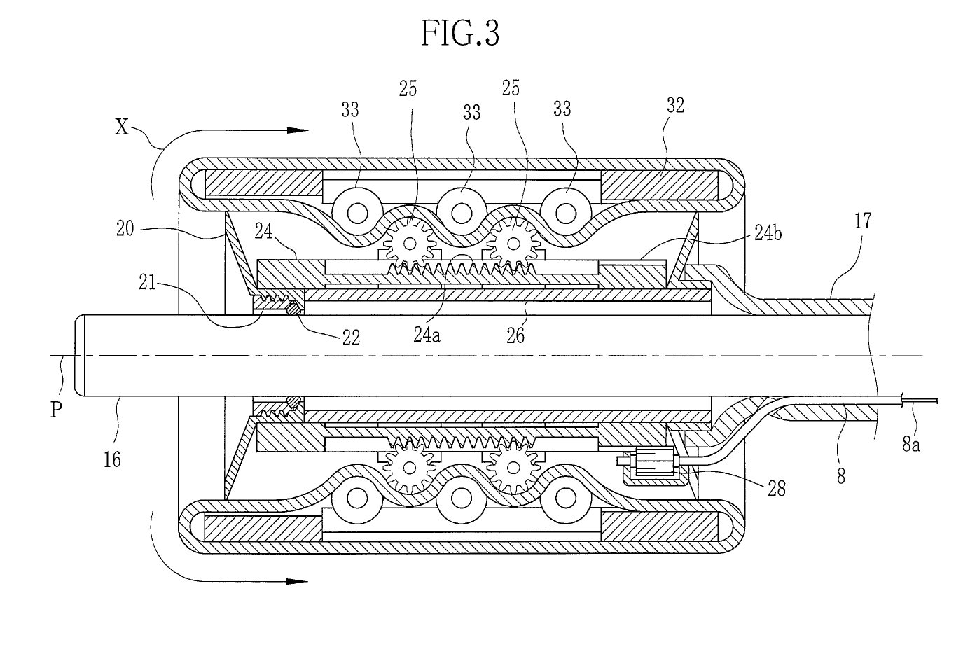

As illustrated in FIG. 2, the promotion unit 6 is attached to the front end side 16 of the insertion section such that a part of the front end projects. A triangular wearing pipe 20 having rounded corners includes a circular wearing hole 20a at the center, and the front end side of insertion section 13 is fitted into the wearing hole 20a. After having decided the project amount of the front end from the promotion unit 6, by screwing a fixation ring 21 on the inner wall of the wearing hole 20a of the wearing pipe 20, the promotion unit 6 is closely fixed to the outer surface of the front end side 16. As illustrated in FIG. 3, at the back of the fixation ring 21, a rubber ring 22 which has a rich elasticity and a small surface frictional resistance is fixed. When the fixation ring 21 is screwed to a thread formed on the inner wall of the wearing pipe 20, the rubber ring 22 is pushed to a receiving plane formed at the back of the wearing pipe 20 with a slant. Since the tilt of the receiving plane is in the direction where the rubber ring 22 which is pushed by screwing the fixation ring 21 is pushed toward the surface of the front end side 16 of the insertion section, as a result, the promotion unit 6 is fixed to the surface of the front end part 16 by clamping power of the rubber ring 22. Note that the method to fix the promotion unit 6 to the front end part 16 of the insertion section is not limited to this, and various kinds of methods can be used.

The promotion unit 6 has the wearing pipe 20 which is fixed to the surface of the front end part 16 as described above, a rotary pipe 24 rotatably supported around the circumference of the wearing pipe 20, and a wheel holding pipe 26 which is integrally connected with the wearing pipe 20 and rotatably holds a pair of worm wheels 25. The rotary pipe 24 has the outer circumferential surface on which a worm gear section 24a and a flat gear section 24b are formed. A pinion 28 engaged in the flat gear section 24b is rotatably supported by the wearing pipe 20, and its central axis is connected to the torque wire 8. As illustrated in FIG. 2, there are three flat sections, each of which is on every 120 degrees around the axis, on the triangular shape wearing pipe 20 of the promotion unit 6, and the above-mentioned pair of worm wheels 25 are provided along the longitudinal direction on each of these flat sections. And each of the pair of worm wheels 25 is engaged with the worm gear section 24a of the rotary pipe 24, so that when the rotary pipe 24 turns around the central axis P, the worm wheel 25 rotates in the forward or backward direction depending on the rotation direction of the rotary pipe 24.

The promotion unit 6 has an inner structure including the above-described wearing pipe 20, rotary pipe 24 and worm wheel 25, and an outer structure that is installed to cover these parts. The outer structure includes a circulation movable body (driving member) 30 composed of a toroid-shaped bag, a support pipe 32 which is enclosed in the circulation movable body 30 to support it from the inside, and three rollers 33 which are rotatably held to the support pipe 32. The inner circumference side of the circulation movable body 30 passes between the pair of worm wheels 25 and the three rollers 33, and the support pipe 32 is installed such that the three rollers 33 press the pair of worm wheels 25. When the worm wheel 25 is turned by a turn of the rotary pipe 24, the circulation movable body 30 held between the rollers 33 moves to a direction of an arrow illustrated in FIG. 2 and FIG. 3. Since the circumference side of the circulation movable body 30 contacts with the body cavity inner wall such as a large intestine, when the circumference side of the circulation movable body 30 moves to the arrow X direction, the driving force to the forward direction is generated at the front end side 16 of the insertion section of the endoscope 2. Note that the fluids such as saline can be filled in the inside of the circulation movable body 30 in the sealed condition to let the circumference side of the circulation movable body 30 closely contact to the body cavity inner wall.

As described above, the insertion assistance function of the promotion unit 6 is obtained by the turn of the rotary pipe 24. The drive of rotary pipe 24 is carried out by a turn of the pinion 28, and the pinion 28 receives rotational force from the outside unit 7 through the torque wire 8 and turns. In this embodiment, since the mechanical rotational force of the motor incorporated in the outside unit 7 is directly transmitted to the pinion 28 by the torque wire 8, it does not need to provide an electrical actuator such as a motor to the promotion unit 6 and becomes advantageous on compactification. Note that when an actuator such as a motor, a solenoid, and a piezoelectric device, which receives an electric signal and performs mechanical displacement, is incorporated in the promotion unit 6, a lead wire may be used in substitution for the torque wire 8 to transmit an electrical turn signal from the outside unit 7.

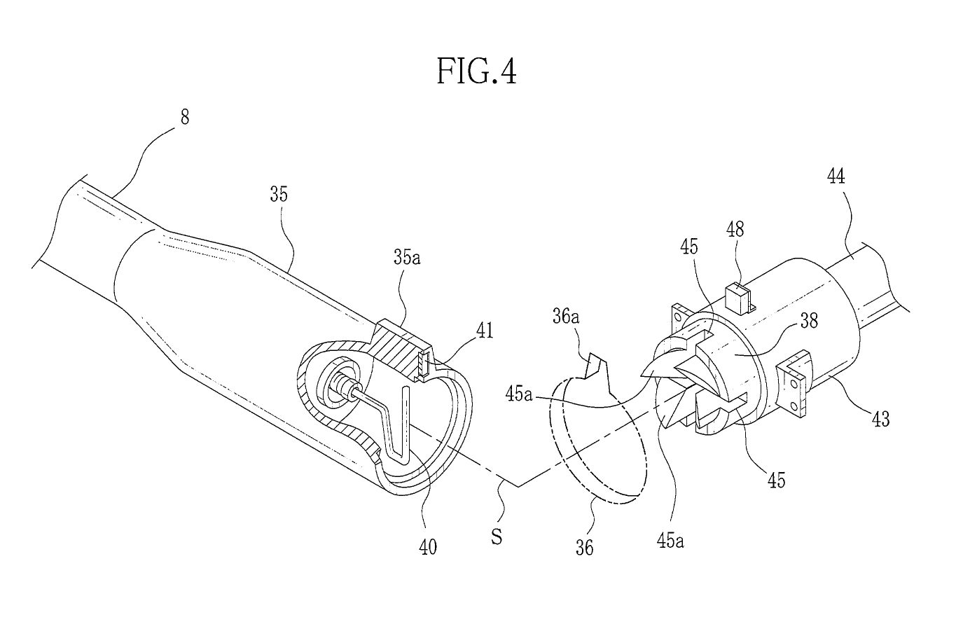

As illustrated in FIG. 1 and FIG. 4, a connector 35 is provided on the rear end of a cover tube of the torque wire 8. The connector 35 is inserted through an opening 36 provided on a front panel of the outside unit 7. Inside of the connector 35, a hook 40 which is connected to a core wire of the torque wire 8 is contained, as illustrated in FIG. 4. And when the hook 40 in the perpendicular posture turns around the axis S, the core wire 8a turns in the same direction in the cover tube of the torque wire 8, and the pinion 28 connected to the end of the wire turns. As a result, the torque wire 8 functions as the transmission section to transmit the rotational force to the promotion unit 6, and the hook 40 becomes the input section receiving the rotational force.

On the upper part of the end of the connector 35, a projection 35a is provided corresponding to a key hole 36a of the opening 36, so that the connector 35 can be inserted into the opening 36 only when the rotational posture of the projection 35a matches the key hole 36a. In addition, an IC tag 41 functioning as the cord given section is adhered on the front end of the projection 35a. The IC tag 41 receives radio waves for reading from an IC tag reader 48, and sends ID information recorded inside it. A code representing a kind of the promotion unit 6 is assigned to this ID information.

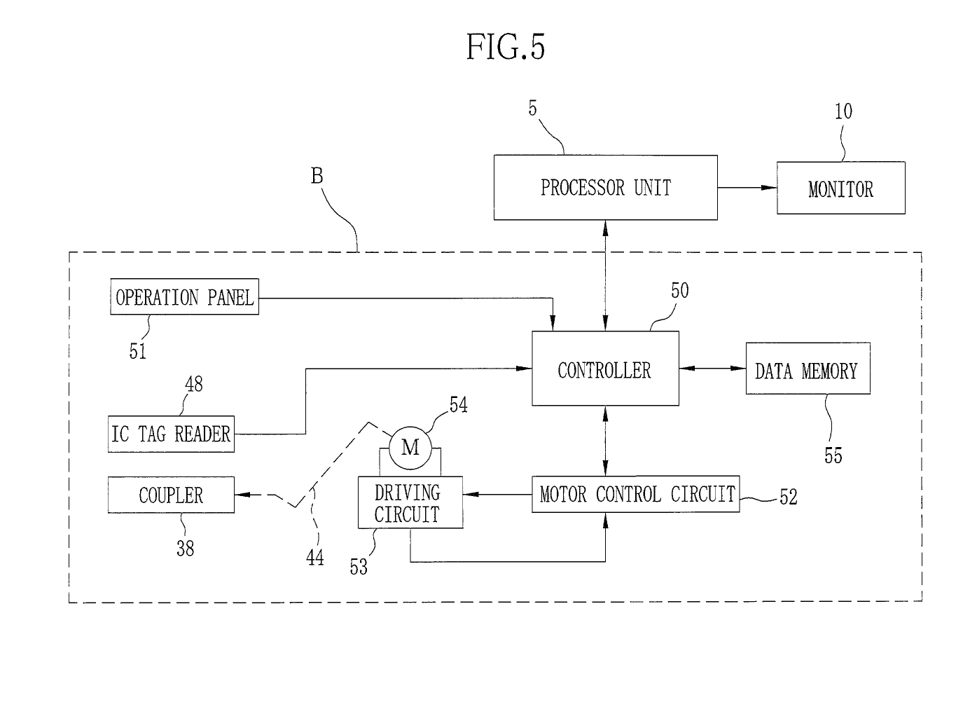

As illustrated in FIG. 4, a sleeve 43 fixed to a housing of the outside unit 7 is provided at the back of the opening 36, and a coupler 38 is rotatably supported by the sleeve 43. At the rear end of the coupler 38, a drive shaft 44, which decelerates and transmits turn of a motor 54 (see FIG. 5) incorporated in the outside unit 7, is connected, and the coupler 38 rotates integrally with the drive shaft 44. The outer diameter of the sleeve 43 almost accords with the inside diameter of the front end of the connector 35, so that when the connector 35 is inserted through the opening 36, the connector 35 is mechanically connected to the sleeve 43 such that a part of the front end of the sleeve 43 is covered. Crossed grooves 45 which are at right angles αach other when these are looked from the front are formed on the front face of the coupler 38, and invitation slopes 45a whose faces are turned by 90 degrees around the axis S are provided in the area sectioned into four parts with the grooves 45.

When the connector 35 is inserted through the opening 36, even if the hook 40 is any rotational posture against the axis S, the hook 40 is rotatably adjusted by operation of the invitation slope 45a, and it fits into either of the linear grooves 45 which are crossed each other. In this way, since the hook 40 is engaged with the coupler 38 so that the core wire 8a of the torque wire 8 is connected to the coupler 38, the rotational force from the motor in the outside unit 7 is transmitted to the pinion 28 through the coupler 38 and the torque wire 8. At this time, the connector 35 is fixed in the state that the leading edge of the sleeve 43 is covered, and the IC tag 41 incorporated in the front end of the projection 35a meets the IC tag reader 48 fixed to the sleeve 43, so that reading of the ID information becomes possible.

In FIG. 5, a block B surrounded in a dashed line B shows an electrical constitution of the outside unit 7. A controller 50 includes a memory where a basic sequence program for the outside unit 7 was written and controls the whole operation of this device. The controller 50 sends control commands for the motor to a motor control circuit 52 according to input operations such as dial operations or button operations through an operation panel 51 at the front face of the housing. The motor control circuit 52 operates a driving circuit 53 in response to an input control command, to control drive of the motor 54. As described above, the turn of the motor 54 is transmitted to the coupler 38 through the decelerating mechanism. In addition, the ID information read by the IC tag reader 48 is input into the controller 50.

The controller 50 accesses a data memory 55 and sends control commands to the motor control circuit 52 based on the read ID information, or communicates signal with the processor unit 5 connected to the outside. As information that is transmitted to the controller 50 from the processor unit 5, there is an identification code representing a kind of the currently connected endoscope 2. In addition, as information sent to the processor unit 5 from the controller 50, there is information about the supporting insertion device, for example ID information of the occupied promotion unit and drive information of the motor 54 which is fed back from the motor control circuit 52, and these information can be displayed with an endoscopic image on the monitor 10 through signal processing in the processor unit 5 side.

By the way, in the system of the present invention, several kinds of the promotion units are prepared, as illustrated as a different type of promotion unit 60 in FIG. 1. The torque wire 8 has the common specification about these promotion units 6 and 60, and as especially for the connector 35 and the hook 40 inside it, these have the same structure. Therefore, either of the promotion units 6 and 60 can be selected to be connected to the outside unit 7. This is because it is necessary for the promotion unit 6 to be changed depending on a kind of the endoscope 2, for example when an outer diameter of the endoscope itself varies between for adult use and for child use, an appropriate one needs to be selected from the promotion units 6 varying in the outer diameter of itself and the inside diameter of the wearing hole 20a. In addition, other than the mechanical dimensions, it is necessary to adjust the drive condition of the circulation movable body 30 which generates driving force for each kind of the promotion unit appropriately.

As an example, when the endoscopes to which one of the insertion assistance devices is applicable are sorted into three kinds of E1 to E3, and the promotion units prepared for these endoscopes are sorted into four kinds of T1 to T4, to the data memory 55 which is incorporated in the outside unit 7, for example, as illustrated in following table 1, data expressing the optimum drive condition for every kind of the promotion unit T1 to T4, and the kind E1 to E3 of the endoscope to which these promotion units cannot be appropriately attached are stored as data.

TABLE 1

| | DRIVE CONDITION OF | | |
|---|---|---|---|
| | PROMOTION UNIT | | |
| KIND OF | VOLTAGE | MAXIMUM | |
| PROMOTION | VARIABLE | CURRENT | INCOMPATIBLE |
| UNIT | RANGE | LEVEL | ENDOSCOPE |
| T1 | V1 to V4 | A1 to A3 | |
| T2 | V1 to V3 | A1 to A2 | |
| T3 | V2 to V4 | A2 to A3 | E1 |
| T4 | V1 to V2 | A1 | E2, E3 |

In table 1 mentioned above, the drive condition of the promotion unit is expressed as the drive condition of the motor 54 which is incorporated in the outside unit 7. For example, the promotion unit of T1 has the variable level of the voltage is V1 to V4, and in this range, an operator can perform change operation through dial control of the operation panel 51 of the outside unit 7 and can regulate the moving speed of the circulation movable body 30. In addition, the maximum torque of the motor can be regulated by setting the maximum current level flowing in the motor 54 within the range of A1 to A4. In addition, a DC motor is used as the motor 54, and the PWM (Pulse Width Modulation) method can be applied for the drive control method of the motor.

Generally, the outer diameter and the axial length of the circulation movable body 30 become different if the kinds of the promotion unit are different, therefore, the diameter and the length of the rotary pipe 24 differ, too. In case that in consideration of production efficiency, gear pitches of the worm gear section 24a and the flat gear section 24b of the rotary pipe 24 are uniform regardless of the kinds, the common pinion 28 is used, and the motor 54 is driven in the same condition, the moving speed of the circulation movable body 30 becomes slower as the rotary pipe 24 becomes to have a larger diameter. Since the promotion unit having a larger diameter of the rotary pipe 24 can be attached to the endoscope having a larger outer diameter, if the motor 54 is driven by rotary signal that the number of revolutions is bigger and the torque is smaller as the promotion unit utilizes the rotary pipe 24 of larger diameter, according to the read code, it becomes able to let the promotion unit generates the driving force within a certain range regardless of the outer diameter of the endoscope.

In this way, by determining the drive condition of table 1 for every kind of the promotion unit so that either one can obtain the appropriate driving force, the operator can operate the operation panel 51 to regulate the drive of the promotion unit within this range. In addition, in the data field of “incompatible endoscope” in table 1, information expressing that the promotion unit of kind T3 cannot be used for the endoscope of kind E1, and that the promotion unit of kind T4 cannot be used for the endoscopes of kinds E1 and E2 is added.

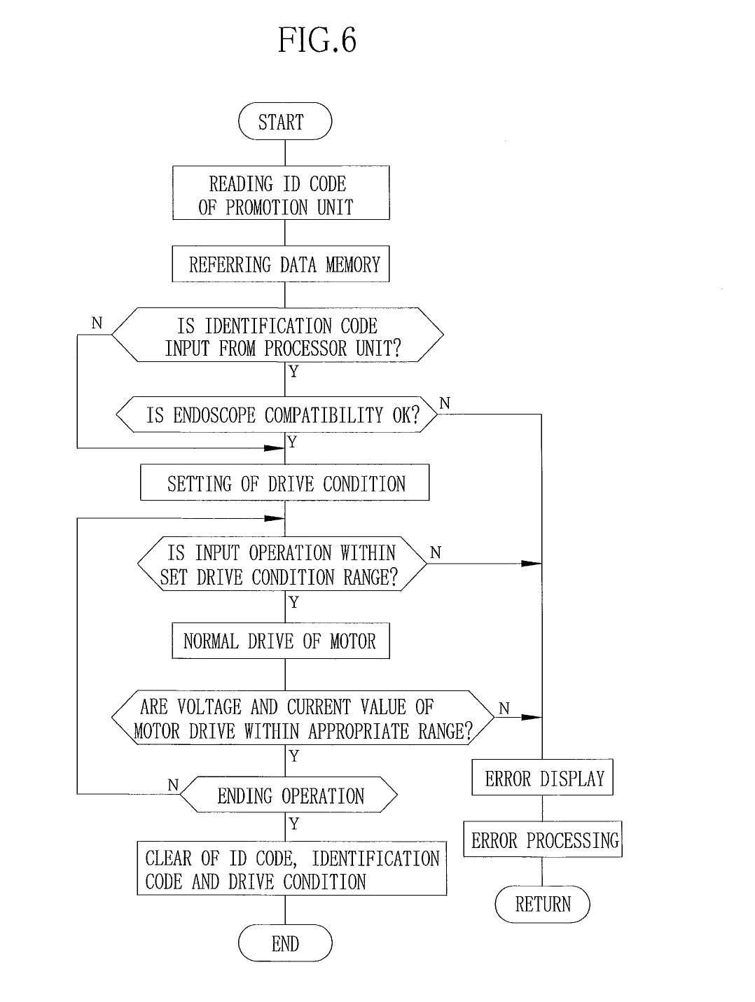

Next, operation of the insertion and extraction assisting device having the above-described construction for the endoscope will be described with reference to FIG. 6. At the time of the use of the insertion and extraction assisting device, at first the front end side 16 of the insertion section of the endoscope 2 is inserted in the wearing hole 20 of the promotion unit 6, and the fixation ring 21 is screwed from the front end side of the wearing pipe 20 to fix the promotion unit 6. The connector 35 of the torque wire 8 drawn out from the endoscope 2 is inserted from the opening 36 of the outside unit 7 to be connected. During this time, the hook 40 connected to the core wire 8a of the torque wire 8 engages the coupler 38, and the rotational force from the drive shaft 44 of motor 54 is transmitted to the pinion 28 through the torque wire 8. Note that these steps and actions are performed in exactly the same way regardless the kind of the promotion unit 6.

The IC tag reader 48 incorporated in the outside unit 7 reads the ID code expressing the kind of the promotion unit 6 from the IC tag 41 fixed to the projection 35a of the connector 35 and inputs it into the controller 50. The controller 50 refers to the data memory 55 and checks whether the identification code expressing the kind of the endoscope 2 is input from the processor unit 5. When the identification code expressing the kind of the endoscope is input, it is compared with the ID code of the promotion unit 6 which is read from the IC tag 41, to judge whether it is compatible. The setting process of the drive condition is executed according to the kind of the promotion unit 6 if it is not incompatible, but if it is confirmed to be incompatible, it is diverged to error processing.

During the above-mentioned processing, the controller 50 sends the reading data of the ID code, the information of there is the identification code input from the processor unit 5 or not, and the result of the compatibility judgment between the promotion unit 6 and the endoscope 2, to the processor unit 5. In this way, the information transmitted from the outside unit 7 is displayed on the monitor 10 through the processor unit 5, so that the operator can easily confirm.

The controller 50 sets the drive condition of the circulation movable body 30 of the promotion unit 6, that is, the drive condition of the motor 54 in the outside unit 7, to the motor control circuit 52. This setting data is also displayed on the monitor 10 in the same way. An operator performs dial operation or button operation on the operation panel 51 while confirming the drive condition through the monitor 10, so that within the drive condition set according to the kind of the promotion unit 6 automatically, the number of revolutions and the maximum torque of the motor 54 can be regulated.

The promotion unit 6 is driven under the permitted drive condition, so that the front end side 16 of the insertion section of the endoscope can be moved in the forward or backward direction for example in a large intestine. When the adjustment operation beyond the range permitted on the drive condition is carried out, an error indication is displayed on the monitor 10, and the input operation is disabled. In addition, while the insertion and extraction assisting device is used, the controller 50 monitors a driving voltage and a motor electric current of the motor 54 fed back from the motor control circuit 52, and these voltage and current value are displayed on the monitor 10 in real time. In this way, whether the motor 54 works normally is judged, and when an abnormal movement was confirmed, an error message is displayed and an emergency shut down as an error handling is executed. This monitoring process is continued until the end operation is carried out.

Under the above-mentioned operation, even if an operator does not memorize the drive condition determined for every kind of the plurality of prepared promotion units, since setting of the range of the drive condition is automatically executed by the outside unit 7, an unprepared setting mistake can be surely prevented. In addition, since the operation states of the motor can be monitored on the monitor 10 continuously during an operation, even when an abnormal movement occurs, it can be quickly handled. Furthermore, under the environment where the endoscope and the insertion and extraction assisting device are systematized, and the identification code expressing the kind of the endoscope is obtained from the processor unit 5, the compatibility between the promotion unit and the endoscope becomes able to be automatically judged.

In the above-described embodiment, mechanical rotational force is transmitted to the promotion unit 6 through the torque wire 8 from the outside unit 7, but the present invention is also applicable in a configuration that an electrical rotary signal is transmitted to the promotion unit 6 from the outside unit 7 to drive a built-in electrical actuator in the promotion unit 6.

In addition, in consideration of careless disconnection, a configuration, that the two torque wires 8 are arranged in parallel and the pinion 28 is connected with the end of the each wire, and the each is engaged with the flat gear section 24b of the rotary pipe 24 to drive in parallel, is effective. Even in this method, the engagement with the coupler 38 is carried out as same as the case that the hook 40 is provided at the other end side of the each torque wire 8 and is connected to the outside unit 7.

As for the present invention, various kinds of transformation, changes without departing from the spirit of the invention are possible, and it should be understood that these are within the protective range of the present invention.

What is claimed is:

1. An insertion and extraction assisting device for an endoscope having an insertion section comprising:
a promotion unit which has a wearing pipe attached on the front end side of the above insertion section and a driving member which is rotatably incorporated in the wearing pipe, and turns the driving member by rotational force or based on a turn signal from outside to give driving force to the insertion section;
a transmission section whose one end side is connected to the promotion unit and the other end side is drawn outside the insertion section, the transmission section having an input section into which the rotational force or turn signal is input, and a cord given section to which a cord expressing the kind of the promotion unit is given, at the other end side; and
an outside unit which includes a coupler to which the input section is connected and inputs the rotational force or turn signal into the transmission section through the coupler, the outside unit further including a reader which reads the cord from the cord given section in the state that the input section is connected to the coupler, and a control section which controls the rotational force or turn signal to be input into the transmission section according to the cord read by the reader.

2. The insertion and extraction assisting device claimed in claim 1, wherein the outside unit includes a memory where a different control signal is assigned according to each of several kinds of the cords, and wherein the above control section controls the rotational force or turn signal based on a control signal obtained with reference to the memory.

3. The insertion and extraction assisting device claimed in claim 2, wherein the above outside unit inputs rotational force or turn signal having a larger number of revolutions into the transmission section as the outer diameter of the insertion section to which the promotion unit is attached is larger.

4. The insertion and extraction assisting device claimed in claim 3, wherein the transmission section has a torque wire which transmits the rotational force, and the outside unit incorporates a motor, rotational force by drive of the motor being input into the input section provided at the other end side of the torque wire through the coupler.

5. The insertion and extraction assisting device claimed in claim 4, wherein the promotion unit is attached such that the circumference of the front end side of the insertion section is surrounded, and the driving member is a toroid-shaped or belt-shaped movable body circulating in the longitudinal direction of the insertion section.

6. An endoscope system comprising:
an endoscope having insertion section and an operation section;
a processor unit to which the endoscope is connected, receiving an image signal obtained from the endoscope and displaying an image on a monitor, and being able to output an identification code according to a kind of the connected endoscope;
a promotion unit which has a wearing pipe attached on the front end side of the above insertion section and a driving member which is rotatably incorporated in the wearing pipe, and turns the driving member by rotational force or based on a turn signal from outside to give driving force to the insertion section;
a transmission section whose one end side is connected to the promotion unit and the other end side is drawn outside the insertion section, the transmission section having an input section into which the rotational force or turn signal is input, and a cord given section to which a cord expressing the kind of the promotion unit is given, at the other end side;
an outside unit which includes a coupler to which the input section is connected and inputs the rotational force or turn signal into the transmission section through the coupler, the outside unit further including a reader which reads the cord from the cord given section in the state that the input section is connected to the coupler, and a control section which controls the rotational force or turn signal to be input into the transmission section according to a combination of the cord read by the reader and the identification code output from the processor unit.

7. The endoscope system claimed in claim 6, wherein the cord read by the readers is input into the processor unit from the outside unit, and the processor unit displays a kind of the promotion unit attached to the front end side of the insertion section of the endoscope, on the monitor.
